(19) United States
(12) Patent Application Publication
(10) Pub. No.: US 2025/0307740 A1
(43) Pub. Date: Oct. 2, 2025

(54) SYSTEMS AND METHODS FOR IDENTIFYING EVENTS IN SUPPLY CHAIN PROCESSES

(71) Applicant: Regeneron Pharmaceuticals, Inc., Tarrytown, NY (US)

(72) Inventors: Fletcher DIX, Clifton Park, NY (US); Diego De La Torre Campos, Albany, NY (US)

(73) Assignee: Regeneron Pharmaceuticals, Inc., Tarrytown, NY (US)

(21) Appl. No.: 19/091,049

(22) Filed: Mar. 26, 2025

Related U.S. Application Data
(60) Provisional application No. 63/570,679, filed on Mar. 27, 2024.

(51) Int. Cl. G06Q 10/0631; A61M 5/20

(52) U.S. Cl. CPC G06Q 10/06315; A61M 5/20; A61M 2205/70; A61M 2207/00

(57) ABSTRACT

A method for identifying an event during a supply chain process that includes preparing a product during the supply chain process, determining an occurrence of the event during preparation of the product during the supply chain process via a sensing device engaged against an interior surface of the product, wherein the event causes the sensing device to disengage the interior surface, thereby indicating a change to the product, and identifying a modification to the supply chain process to inhibit occurrences of the event during subsequent preparations of the product in the supply chain process. The event causing a change of the product.

### CROSS-REFERENCE TO RELATED APPLICATIONS

This application claims priority to U.S. Provisional Patent Application No. 63/570,679, filed on Mar. 27, 2024, which is hereby incorporated by reference in its entirety.

### FIELD OF DISCLOSURE

Aspects of the present disclosure relate to systems and methods for identifying an occurrence of an event during a supply chain process, such as for a product. More specifically, embodiments of the present disclosure relate to systems and methods for detecting the occurrence of an event and determining one or more measurable parameters (e.g., a force, a pressure, a temperature, a light intensity, an impedance, etc.) experienced by a product at a particular stage of a supply chain process. The product may include an apparatus or a device, such as a medical device. The supply chain process may include one or more stages, such as manufacturing, assembly, testing, sterilizing, packaging, and/or delivery of the product.

The systems and methods of the present disclosure may identify a particular stage during the supply chain process, such as one or more periods and/or points of time, in which the event occurs. The event may be indicative of an error, inefficiency, or fault in the supply chain process at which the product may become structurally compromised, damaged, or rendered inoperable for use. The source of the event may be attributed to various characteristics or sources in the supply chain process, such as instrumentation and/or tools used for preparing and/or managing the product during the particular stage at which the event occurs. By identifying the stage of the supply chain process in which the event occurs, the systems and methods of the present disclosure may identify a modification of one or more of the characteristics or sources associated with the particular stage of the supply chain process to mitigate future occurrences of the event.

### INTRODUCTION

A preparation and/or management of a product during a supply chain process may involve manufacturing, assembling, testing, sterilizing, packaging, and/or delivering the product (e.g., a medical device, such as a syringe storing a fluid substance) in various arrangements, positions, configurations, and/or locations. In some instances, a parameter, such as a load (e.g., a force or an acceleration event), may be inadvertently applied to the product by one or more sources associated with the supply chain process, such as manufacturing instrumentation. Application of the parameter may cause unintended modifications and/or damage to the product. In instances where the product is a medical device, and more particularly a syringe (or a vial, cartridge, or otherwise a glass primary container), such modifications and/or damage may result in premature failures and/or inaccurate use of the syringe by a user. In other instances, human error in handling, controlling, positioning, and/or delivering the product (e.g., a syringe) during a stage of the supply chain process may similarly entail the application of a parameter that may influence a structural integrity of the product.

The occurrence (i.e., the event) of a parameter (e.g., a load) being applied to a syringe prior to its use in administering a dose to a user may cause health and safety complications for the user (e.g., a patient). For example, occurrences of a load to the syringe may result in a misalignment of a needle of the syringe, an inadvertent release of at least a portion of a fluid substance stored in the syringe, breakage of a material (e.g., glass, plastic, etc.) forming a container or vial of the syringe storing the fluid substance, and more, thereby resulting in syringes that are ineffective for potential use by users.

### SUMMARY

Disclosed herein are systems and methods for identifying an event during a supply chain process of a product, and particularly medical devices such as syringe containers or vials, storing a fluid substance, or a lyophilized product, including, but not limited to, substances that are in a powdered or freeze dried form, in which the event changes and/or causes damage to the product. In one embodiment of the present disclosure, a method for identifying an event during a supply chain process includes preparing a product during the supply chain process; determining an occurrence of the event during preparation of the product during the supply chain process, wherein the event causes a change of the product and/or at least partially impairs the product; and identifying a modification to the supply chain process to inhibit occurrences of the event during subsequent preparations of the product. The occurrence of the event may be determined by one or more sensing devices disposed within an interior portion the product, and particularly within a cavity of a syringe container or vial.

When such sensing devices are disposed along an exterior surface of the product, one or more parameters applied to the product may be inadvertently undetected and/or inaccurately measured, such as when the product (e.g., syringe container or vial) is positioned inside a packaging or an outer device body (e.g., an auto-injector). In these instances, the sensing devices may be at least partially ineffective and/or inoperable for determining an occurrence of an event during the supply chain process.

In other instances, with the sensing devices having surface properties or characteristics that differ from that of the product, the sensing device may generate a modified exterior interface on the product that interacts with the supply chain process in a manner that varies from an original exterior surface of the product. For example, the sensing devices may be formed of a first material (e.g., plastic, rubber, etc.) that varies from a second material (e.g., glass) of the product (e.g., a syringe container or vial), such that a parameter applied to the modified exterior interface of the product may not be accurately representative of a true parameter experienced by the product when the sensing devices are omitted.

In further instances, with the one or more sensing devices positioned along the exterior of the product, such sensing devices may form a greater cross-sectional profile of the product relative to an original cross-sectional dimension of the product. The increased size of the product with the sensing devices attached thereto may influence a degree to which a parameter is applied to the product and/or detected by the sensing devices. Additionally and/or alternatively, a relative size of a packaging and/or an outer device body may require modifications to accommodate the increased size of the product, which may further contribute to inaccurate measurements of the true parameter applied to the product. By disposing one or more sensing devices within the interior of the product (i.e. a syringe container or vial), the one or more sensing devices may be configured to accurately detect a parameter applied to the product, thereby improving the determination of an occurrence of an event during the supply chain process. accurate

### BRIEF DESCRIPTION OF THE DRAWINGS

The accompanying drawings, which are incorporated into and constitute a part of this specification, illustrate various exemplary embodiments and, together with the description, serve to explain principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements in various embodiments, other than those specifically shown, are contemplated and are within the scope of the present disclosure.

FIG. 1 depicts an exploded elevation view of an exemplary product including a medical device with an example sensing device disposed within the medical device, according to aspects of the present disclosure.

FIG. 2 depicts a side elevational view of an exemplary container of the medical device of FIG. 1, with the sensing device disposed inside the container, according to aspects of the present disclosure.

FIG. 3A depicts a side elevational view of an exemplary vial of the medical device of FIG. 1, with the example sensing device disposed inside the vial, according to aspects of the present disclosure.

FIG. 3B depicts a side elevational view of an exemplary cartridge of the medical device of FIG. 1, with the example sensing device disposed inside the vial, according to aspects of the present disclosure.

FIG. 4 depicts a schematic of an exemplary supply chain process of the product of FIG. 1, according to aspects of the present disclosure.

FIG. 5 depicts a flowchart of an exemplary method for identifying an occurrence of an event to the product of FIG. 1 during the supply chain process of FIG. 4, according to aspects of the present disclosure.

FIG. 6 depicts a schematic of an exemplary manufacturing stage of the supply chain process of FIG. 4, according to aspects of the present disclosure.

FIG. 7 depicts a perspective view of another exemplary vial of the medical device of FIG. 1 including an attachment mechanism for securing sensing devices within the vial, according to aspects of the present disclosure.

FIG. 8 depicts a side elevational view of another exemplary vial of the medical device of FIG. 1 including an attachment mechanism for securing sensing devices within the vial, according to aspects of the present disclosure.

FIG. 9 depicts the attachment mechanism of FIG. 8 securing the sensing device to the interior surface of the vial, according to aspects of the present disclosure.

FIG. 10 depicts another exemplary attachment mechanism securing the sensing device to the interior surface of the vial of FIG. 8, according to aspects of the present disclosure.

FIG. 11 depicts another exemplary attachment mechanism securing the sensing device to the interior surface of the vial of FIG. 8, according to aspects of the present disclosure.

FIG. 12 depicts another exemplary attachment mechanism securing the sensing device to the interior surface of the vial of FIG. 8, according to aspects of the present disclosure.

There are many embodiments described and illustrated herein. The present disclosure is neither limited to any single aspect nor embodiment thereof, nor to any combinations and/or permutations of such aspects and/or embodiments. Each of the aspects of the present disclosure, and/or embodiments thereof, may be employed alone or in combination with one or more of the other aspects of the present disclosure and/or embodiments thereof. For the sake of brevity, many of those combinations and permutations are not discussed separately herein.

### DETAILED DESCRIPTION

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Notably, an embodiment or implementation described herein as an “example” or “exemplary” is not to be construed as preferred or advantageous, for example, over other embodiments or implementations; rather, it is intended reflect or indicate the embodiment(s) is/are one “example,” rather than “ideal.” In addition, the terms “first,” “second,” and the like, herein do not denote any order, quantity, or importance, but rather are used to distinguish an element, a structure, a step or a process from another. Moreover, the terms “a” and “an” herein do not denote a limitation of quantity, but rather denote the presence of one or more of the referenced items. Additionally, the terms “about,” “approximately,” “substantially,” and the like, when used in describing a numerical value, denote a variation of +/−10% of that value, unless specified otherwise.

Embodiments of the present disclosure may be used for any type of product, including fluid-containing products, such as liquid drug substances, liquid placebos, other liquids (or liquid solutions), or a lyophilized product, including, but not limited to, substances that are in a powdered or freeze dried form that may be dispensed in a dose form. As used herein, the term “substance” may refer to a formulated substance including an active ingredient or ingredients. In some embodiments, systems and aspects of the present disclosure can be used for apparatuses and/or devices manufactured for use with any therapies using the immune system to combat diseases, such as immunotherapies, including vaccines, allergy treatments, cancer treatments, and more. In particular, systems and aspects of the present disclosure can be used for apparatuses and/or devices manufactured for the treatment of patients with cancer (e.g., immune-oncology). However, it is also contemplated that embodiments of the present disclosure may be applicable to apparatuses and/or devices used for any therapy, liquid products, and/or any other context for which one or more substances may be stored. While medical indications are described herein, it is contemplated that non-medical apparatuses and/or devices may be used in conjunction with the systems and methods described herein. It should be understood that embodiments of the present disclosure may be used with apparatuses and/or devices that do not contain any liquid substance.

As used herein, the terms “distal” and “distally” refer to a location (or portion of a device) relatively closer to, or in the direction of, a drug delivery end of the device, and the terms “proximal” and “proximally” refer to a location (or portion of a device) relatively closer to, or in the direction of, an end of the device opposite the distal end. As used herein, the term “component,” when used in reference to a part of a product, may refer to a feature of the product suitable for serving any purpose of the product. In examples in which the product may include a medical device, the component may include a body, e.g., a barrel (such as a syringe barrel, a syringe container, a vial), tube, cylinder, or other portion of a device capable of storing a substance. In some embodiments, a body may also include a distal end portion having a nozzle, needle, needle attachment site, and/or distal cap. In other embodiments, a body may include a proximal end portion having a valve, a seal, and/or proximal cap.

Described herein is an exemplary embodiment of a product, such as an apparatus, and in particular a medical device such as an auto-injector including a syringe container. In some instances, embodiments or aspects of embodiments disclosed herein may be used with medical devices including other types of substance-containing products, such as, but not limited to, vials (see FIGS. 3A and 3B), cartridges, and/or other suitable devices. In the examples, a component of the medical device, and in particular a syringe container and/or a vial, may be formed of glass or plastic. In further instances, embodiments or aspects of embodiments disclosed herein may be used with various other suitable products (e.g. apparatuses and/or devices). Such systems and methods may aid to identify sources of human or automated equipment error in a supply chain process, and/or increase accuracies and efficiencies in a supply chain process, by identifying an occurrence of an event during the supply chain process, such as during one or more stages of said process (e.g., a manufacturing, an assembly, a testing, a sterilizing, a packaging, a labeling/marking, a storage, a shipment, a sale, and/or a delivery process of the product).

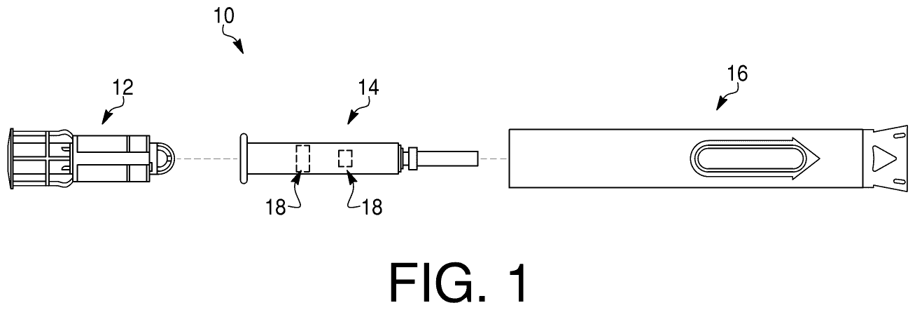

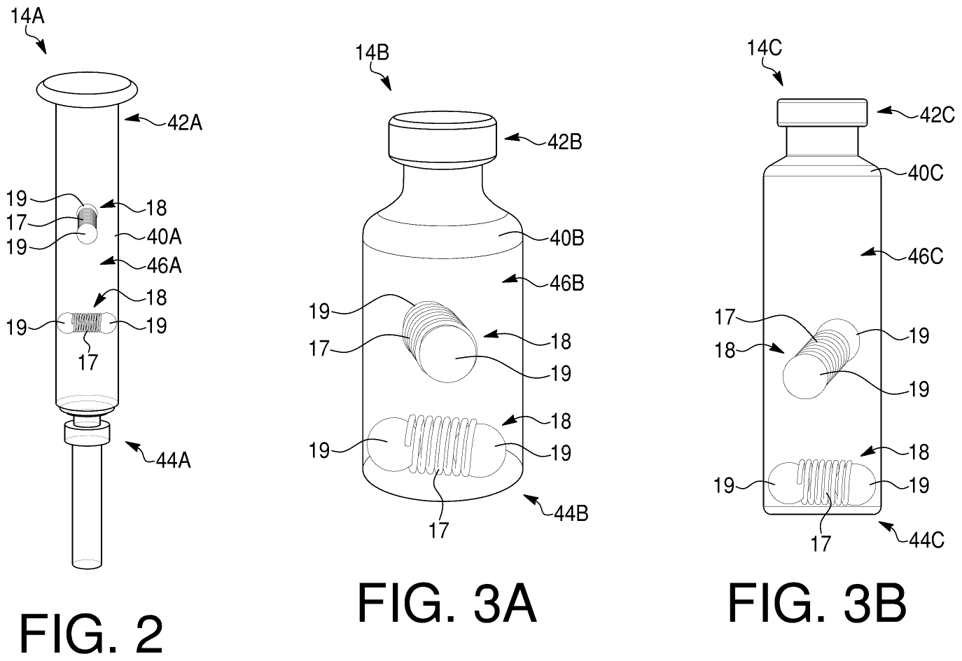

Referring now to FIG. 1, an exemplary product 10 is depicted. It should be appreciated that product 10 may include various manufactured goods, including but not limited to, a device, a tool, a machine, an instrument, a mechanism, an appliance, a gadget, an apparatus, or other various equipment. By way of example only, product 10 may include a medical device (e.g., an auto-injector) including a plurality of components, such as a first component 12, a second component 14, and a third component 16. First component 12 may include a cap that is configured to selectively couple with third component 16 to enclose one or more contents therein. Second component 14 may include a container configured to store a substance, such as a medicament (e.g. a drug product). In some embodiments, the container of second component 14 may include, but is not limited to, a syringe (see FIG. 2), a vial (see FIG. 3A), a barrel, a cartridge (see FIG. 3B), a tube, and more.

Third component 16 may include a housing that is sized, shaped, and/or otherwise configured to at least partially receive one or more of first component 12 and second component 14 therein. In other words, third component 16 may define an outer body of product 10 for storing the internal components of product 10, such as first component 12 and second component 14. Each of first component 12, second component 14, and third component 16 may be formed of various suitable materials, including but not limited to, glass, plastic, etc. For example, first component 12 may be formed of a first material, second component 14 may be formed of a second material, and third component 16 may be formed of a third material. In some instances, the first and third materials may be substantially similar to one another, and the second material may be different from the first and third materials. In one example, the first and third materials may include a plastic, and the second material may include glass.

It should be understood that product 10 (e.g., an auto-injector) may include additional and/or fewer components than that shown and described herein, including but not limited to, a needle, a plunger, a biasing mechanism, and more. Alternatively, first component 12, second component 14, and/or third component 16 of product 10 may include various other shapes, sizes, arrangements, or configurations than those shown and described herein without departing from a scope of this disclosure. As shown and described in further detail below, second component 14 may be sized, shaped, and/or otherwise configured as a container, a vial, and/or various other suitable devices (see FIGS. 2-3). In some embodiments, one or more of first component 12, second component 14, and third component 16 may be omitted from product 10 entirely.

Still referring to FIG. 1, one or more sensing devices 18 may be coupled to product 10, and particularly to one or more of first component 12, second component 14, and/or third component 16. It should be understood that sensing device(s) 18 may be selectively coupled to product 10, such that sensing device(s) 18 are not a component of product 10. Stated differently, sensing device(s) 18 may be removably attached to product 10 for purposes of identifying the occurrence of an event during a supply chain process of product 10 according to aspects of the present disclosure, such that sensing device(s) 18 are not an ordinary feature of product 10. In the example, product 10 may include a pair of sensing devices 18. Moreover, though only a pair of sensing devices 18 are depicted, those of ordinary skill will understand that any suitable number of sensing devices 18 may be provided within the scope of this disclosure. For example, one, three, four, or any suitable number of sensing devices 18 may be provided, according to the scope of this disclosure.

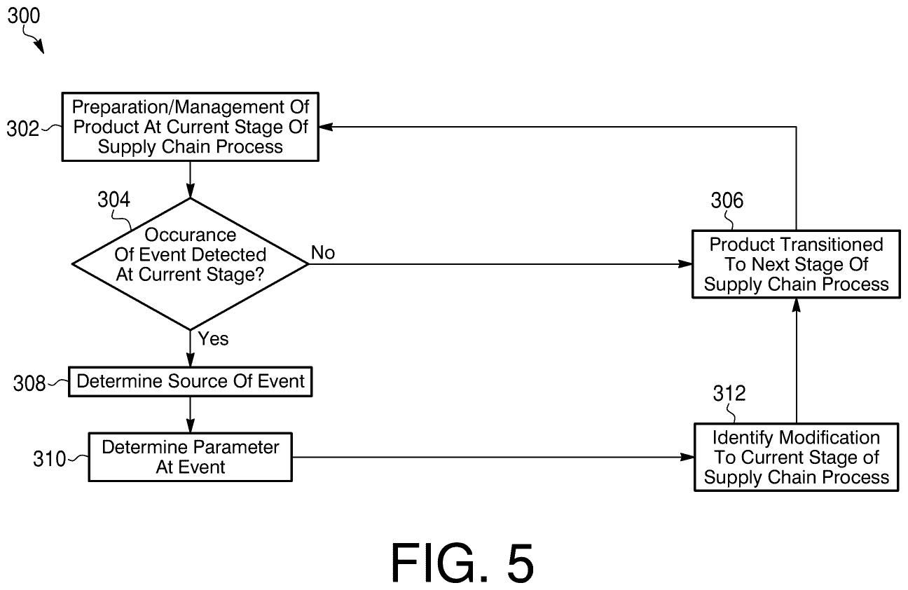

Sensing devices 18 may be coupled to and/or inserted within second component 14 in the present example. In other examples, sensing devices 18 may be positioned on and/or within first component 12 and/or third component 16 in lieu of and/or in addition to second component 14. It should be appreciated that including sensing devices 18 in product 10 may render product 10 as a “test” or “experimental” version of product 10 for administering the methods described in further detail herein (see FIG. 5). In other embodiments, sensing devices 18 may be an ordinary feature of product 10.

FIG. 2 depicts a side elevational view of an exemplary second component 14A of product 10 of FIG. 1. It should be appreciated that second component 14A may be incorporated into product 10 in lieu of second component 14 shown and described above without departing from a scope of this disclosure. In the example, second component 14A may include a syringe container having a body 40A with a longitudinal length defined between a proximal end 42A and a distal end 44A. Second component 14A may include an internal cavity 46A defined by body 40A between proximal end 42A and distal end 44A for storing a fluid substance. Body 40A of second component 14A may be formed of a (second) material, such as glass. Second component 14A may include a pair of sensing devices 18 disposed inside internal cavity 46A. Each of the pair of sensing devices 18 may include a biasing mechanism 17 (e.g., a spring), and a pair of retention mechanisms 19 (e.g., balls) disposed along opposing ends of biasing mechanism 17. In embodiments where the retention mechanisms 19 are balls, the balls may be made of any suitable material, including, but not limited to, steel or plastic. Biasing mechanism 17 may be biased towards an expanded configuration, such that biasing mechanism 17 may be configured to urge the pair of retention mechanisms 19 relatively away from one another.

In some embodiments, each of the pair of retention mechanisms 19 may include and/or be formed of copper contacts, such that biasing mechanism 17 may be configured to separate and/or hold the copper contacts at retention mechanisms 19 apart from one another. In this instance, sensing device 18 may be configured and operable to create an open circuit. Sensing devices 18 may be disposed inside internal cavity 46A with each of the pair of retention mechanisms 19 in abutting contact with an interior surface of body 40A of second component 14A. It should be appreciated that a diameter of internal cavity 46A of body 40A may be relatively less than a longitudinal length of sensing device 18, and particularly biasing mechanism 17 when in the expanded configuration, such that biasing mechanism 17 may be transitioned to a partially compressed configuration when disposed in internal cavity 46A.

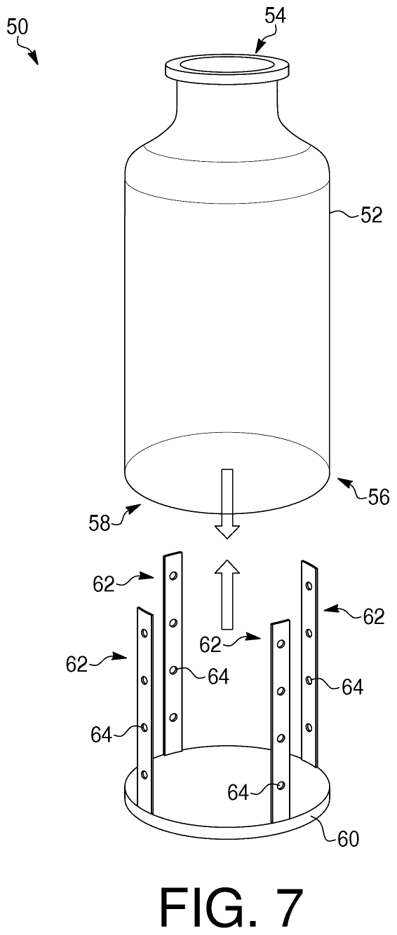

As described above, the pair of retention mechanisms 19 of sensing devices 18 may be biased radially-outwards from one another by biasing mechanism 17 disposed there between, such that sensing devices 18 may be fixedly coupled to second component 14A at a fixed position by biasing mechanism 17 pushing retention mechanisms 19 against the interior surface of second component 14A. In other words, a relative position, location, and/or orientation of each sensing device 18 within body 40A may be fixed in response to biasing mechanism 17 applying a radially outward force onto each of the pair of retention mechanisms 19 against the internal surface of body 40A. As such, sensing devices 18 may be fixedly coupled to body 40A due to biasing mechanism 17 urging retention mechanisms 19 against the internal surface of body 40A with sufficient force to securely maintain a position, a location, and/or an orientation of sensing device 18 within internal cavity 46A. In some embodiments, sensing devices 18 may be adhered to the internal surface of body 40A to securely retain sensing devices 18 to the fixed position, such as, for example, via an epoxy. In other embodiments, as described herein, the internal surface may include one or more attachment mechanisms for securely coupling sensing devices 18 thereto (see FIGS. 7-12).

Still referring to FIG. 2, sensing devices 18 may be configured such that one or more of the pair of retention mechanisms 19 may move upon the occurrence of an event to product 10, and particularly to second component 14A, such as an application of force that is sufficient to cause retention mechanisms 19 to move relative to body 40A and/or cause biasing mechanism 17 to compress and/or expand between the pair of retention mechanisms 19. In this instance, at least one of the pair of sensing devices 18 may become dislodged from the fixed position within internal cavity 46A, thereby decoupling the at least one sensing device 18 from 3 engagement with the internal surface of body 40A. In some embodiments, a user of product 10 may be able to detect the occurrence of the event by visually inspecting second component 14A to determine whether one or more sensing devices 18 have moved relative to its original, fixed position within internal cavity 46A.

It should be appreciated that product 10 may be configured and operable to provide detection of a parameter (e.g., force) in multiple directions due to the configuration of the pair of sensing devices 18 within body 40A set at a 90° (degree) angle from each other, such as a first (x) direction and a second (y) direction. This configuration of the pair of sensing devices 18 may be configured to allow product 10 to register a parameter (e.g., an impact of force) from multiple angles with enhanced accuracy. In some embodiments, the inclusion of the pair of sensing devices 18 in the configuration shown and described herein may allow for detecting and/or determining additional data points indicative of the occurrence of an event, thus further enhancing an accuracy of a force detection by product 10. In other embodiments, product 10 may include additional sensing devices 18 than those shown and described herein to detect and/or determine further data points for confirming the occurrence of an event.

In one embodiment, and as described in further detail below, sensing devices 18 may be communicatively coupled to a remote system or computer, such as via one or more wires and/or via a wireless connection. The remote system may define an open circuit with the pair of sensing devices 18, and may be configured to complete and/or close the circuit upon at least one of the sensing devices 18 becoming dislodged within body 40A. In this instance, sensing device(s) 18 may be configured to generate an alert, a warning, and/or a notification of the application of force onto product 10. Product 10 may be configured such that only one of the pair of sensing devices 18 may move and/or become dislodged to close the circuit. In another embodiment, as described above, a resulting position of sensing device(s) 18 may be determined upon a visual inspection of product 10, such that sensing device(s) 18 may notify a user of the occurrence of an event based on visual feedback of its dislodged state within body 40A.

Each of the pair of sensing devices 18 may be configured to detect and/or measure one or more parameters indicative of a characteristic and/or measurable parameter experienced by product 10. For example, sensing devices 18 may include a force sensor that is configured to detect and/or measure a force load applied to product 10. In another example, sensing devices 18 may include an accelerometer configured to detect and/or measure an acceleration, agitation, spinning, and/or vibration of product 10. By way of further example, sensing devices 18 may include a gyroscope configured to detect and measure an angular velocity of product 10. In other examples, sensing devices 18 may include a pressure (e.g., piezoelectric) sensor. It should be understood that sensing devices 18 may be configured to detect and/or measure various other suitable parameters (e.g. a rotation, a tilt, a compression, etc.) than those described herein without departing from a scope of this disclosure.

Sensing devices 18 may include a plurality of sensors disposed on one or more components of product 10, with each of the plurality of sensors being configured to detect and measure the same or different parameters. For example, sensing devices 18 may include a sensor array having one or more sensors that may be coupled to a single or multiple components of a product. In one embodiment, the sensor array may include approximately 16 to 32 sensors. In other embodiments, a single sensing device 18 may be configured and operable to detect and measure two or more parameters. In the embodiment, sensing devices 18 may be configured and operable to detect and/or measure a parameter (e.g., a force) with an accuracy of approximately +/−25%, approximately +/−20%, approximately +/−15%, approximately +/−10%, approximately +/−5%, approximately +/−2%, approximately +/−1%, approximately +/−0.5%. In some embodiments, sensing devices 18 may be configured and operable to detect and/or measure a parameter (e.g., a rated G-force) ranging from approximately 15 g to approximately 300 g, and particularly from approximately 20 g to approximately 100 g. For example, sensing devices 18 may become dislodged from the fixed position within body 40A upon product 10 encountering a parameter that is within and/or exceeds a preset G-force range. In one example when product 10 includes a total weight of 40 lbs. (pounds) or less, the rated G-force may range from approximately 80 g to approximately 300 g.

In another embodiment, sensing devices 18 may be configured and operable to detect and/or measure a parameter (e.g., a force) with an accuracy of approximately +/−25%, approximately +/−20%, approximately +/−15%, approximately +/−10%, approximately +/−5%, approximately +/−2%, approximately +/−1%, approximately +/−0.5%. In this embodiment, sensing devices 18 may be configured and operable to detect and/or measure a parameter (e.g., a rated G-force) ranging from approximately 10 g to approximately 500 g, and particularly from approximately 25 g to approximately 100 g. It should be appreciated that sensing device(s) 18 may be configured with various characteristics and properties without departing from a scope of this disclosure. For example, sensing device(s) 18 may include a spring constant that determines an impact (force) threshold that is indicative of the occurrence of a triggering event. In other words, a spring tension of sensing device(s) 18 may correspond to a minimum acceleration force necessary to trigger the occurrence of an event for moving sensing device(s) 18 within body 40A.

FIG. 3A depicts a side elevational view of another exemplary second component 14B of product 10 of FIG. 1. It should be appreciated that second component 14B may be incorporated into product 10 in lieu of second component 14, or 14A shown and described above without departing from a scope of this disclosure. In the example, second component 14B may include a vial having a body 40B with a longitudinal length defined between a proximal end 42B and a distal end 44B. Second component 14B may include an internal cavity 46B defined by body 40B between proximal end 42B and distal end 44B for storing the fluid substance. Body 40B of second component 14B may be formed of a (second) material, such as glass and/or plastic. Second component 14B may include a pair of sensing devices 18 disposed inside internal cavity 46B.

Sensing devices 18 may be in contact with the interior surface of body 40B of second component 14B. Sensing devices 18 may be biased radially-outwards as described in further detail above, such that sensing devices 18 may be fixedly coupled to second component 14B at a fixed position. It should be appreciated that product 10, and particularly the pair of sensing devices 18, may be configured and operable in a substantially similar manner as described above when second component 14B includes the vial in lieu of the syringe container (FIG. 2). In some embodiments, sensing devices 18 may be adhered to the internal surface of body 40B to securely retain sensing devices 18 to the fixed position, such as, for example, via an epoxy. In other embodiments, second component 14B may include one or more attachment mechanisms for securely coupling sensing devices 18 to the interior surface of body 40B (see FIGS. 7-12).

FIG. 3B depicts a side elevational view of another exemplary second component 14C of product 10 of FIG. 1. It should be appreciated that second component 14C may be incorporated into product 10 in lieu of second component 14, 14A, or 14B shown and described above without departing from a scope of this disclosure. In the example, second component 14C may include a cartridge having a body 40C with a longitudinal length defined between a proximal end 42C and a distal end 44C. Second component 14C may include an internal cavity 46C defined by body 40C between proximal end 42C and distal end 44C for storing a variety of substances including but not limited to fluid, gel, powder, etc. Body 40C of second component 14C may be formed of a (second) material, such as glass and/or plastic. Second component 14C may include a pair of sensing devices 18 disposed inside internal cavity 46C.

Sensing devices 18 may be in contact with the interior surface of body 40C of second component 14C. Sensing devices 18 may be biased radially-outwards as described in further detail above, such that sensing devices 18 may be fixedly coupled to second component 14C at a fixed position. It should be appreciated that product 10, and particularly the pair of sensing devices 18, may be configured and operable in a substantially similar manner as described above when second component 14C includes the cartridge in lieu of the syringe container (FIG. 2). In some embodiments, sensing devices 18 may be adhered to the internal surface of body 40C to securely retain sensing devices 18 to the fixed position, such as, for example, via an epoxy. In other embodiments, second component 14C may include one or more attachment mechanisms for securely coupling sensing devices 18 to the interior surface of body 40C (see FIGS. 7-12).

Although product 10 is shown and described herein as a medical device, and particularly an auto-injector, that has a particular configuration and arrangement of assembled components, it should be understood that product 10 may include various other medical devices, medical packages, or non-medical apparatuses such that the systems and methods described herein are not limited to auto-injectors. By way of illustrative example only, other exemplary medical products may include, e.g., suppository applicators and medication, transdermal drug delivery devices, medical implants, needles, cannulas, medical instruments, blister packs, boxes, custom primary containers, vials, cartridges, and more.

Still referring to FIG. 1, and as described in detail herein, sensing devices 18 may be operable to measure a parameter(s) experienced by product 10 from one or more sources (e.g., objects, surfaces, etc.) located in a surrounding environment of product 10 during a supply chain process of product 10. The parameter may cause a corresponding defect or damage to product 10, such as to one or more subcomponents of product 10 (e.g., second component 14, second component 14A, second component 14B, etc.), and may originate from one or more sources that interact with product 10 during its preparation and/or management in the supply chain process.

In some embodiments, sensing devices 18 may be coupled to one or more electronic capacitors including a timer device (not shown). The timer device may be configured and operable to commence timing when the electrodes begin touching one another at the beginning of a supply chain process. Upon the occurrence of an event, one or more of sensing devices 18 may become dislodged and a circuit between the electrodes may break and/or disconnect as a result, thereby causing the timer device to cease timing. In this instance, a user of product 10 may be able to determine the occurrence of the event to product 10 and a corresponding time point of the occurrence relative to a total duration of the supply chain process from the elapsed time recorded by timer device. It should be appreciated that sensing devices 18 may include a battery (not shown) configured and operable for powering the electronic capacitor(s) and/or timer device.

In some embodiments, sensing devices 18 may be operable to communicate with one or more remote systems or computers via various suitable communication protocols. For example, sensing devices 18 may be operable to communicate with a remote system or computer (not shown) via a network that may be implemented as a wireless network (e.g. Wi-Fi), a wired network (e.g., Ethernet), a local area network (LAN), a Wide Area Network (WANs), Bluetooth, Near Field Communication (NFC), cellular satellite, or any other type of network(s) capable of providing communication between sensing devices 18 and a remote system.

The remote system or computer may be configured and operable to carry out one or more steps of the methods shown and described herein according to the present disclosure, such as method 300 of FIG. 5. Although not shown, it should be understood that the remote system may include one or more computer hardware platforms having a central processing unit (“CPU”) in the form of one or more processors for executing computer-readable instructions according to the exemplary embodiments of the present disclosure. The one or more computer hardware platforms may also include a data storage for various data files to be processed and/or communicated, such as data from sensing devices 18. In other embodiments, sensing devices 18 is not in communication with any remote systems or computers such that a user of product 10 may carry out the steps of method 300.

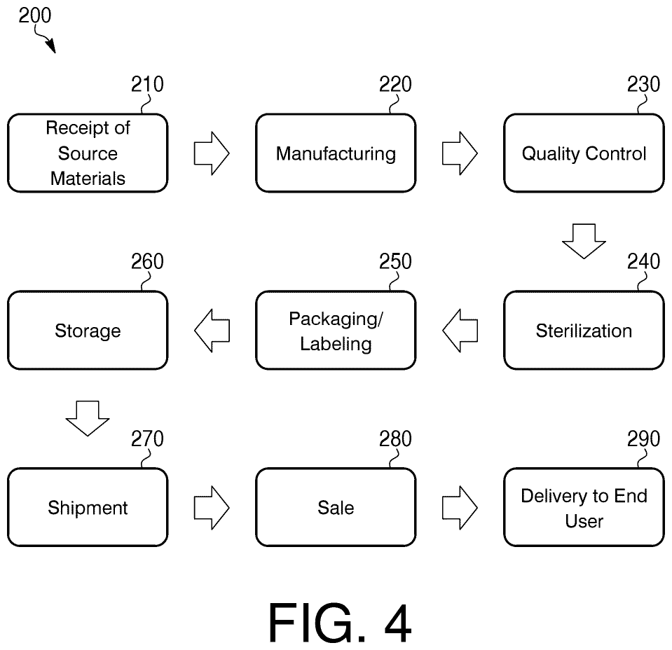

Referring now to FIG. 4, an exemplary supply chain process 200 of product 10 is depicted. Supply chain process 200 (hereinafter “process 200”) may include one or more stages or sub-processes as shown and described herein. It should be appreciated that the stages of process 200 are merely illustrative, such that additional and/or fewer stages may be included in process 200 without departing from a scope of this disclosure. Additionally, some stages of process 200 may be omitted, combined, and/or performed in a different order than that shown and described herein while remaining consistent with this disclosure. Although process 200 is described herein in reference to a single product 10, it should be appreciated that process 200 may involve a plurality of products 10.

In the example, process 200 may include a plurality of stages, such as a first stage 210 involving receipt of source materials for producing product 10; a second stage 220 involving a manufacture and/or assembly of product 10; a third stage 230 involving a quality control testing (or other forms of testing) of product 10; a fourth stage 240 involving a sterilization or cleaning of product 10; a fifth stage 250 involving a packaging and/or labeling of product 10; a sixth stage 260 involving a storage of product 10; a seventh stage 270 involving a shipment of product 10; an eight stage 280 involving a sale of product 10; and a ninth stage involving a delivery of product 10, such as to an end user.

By way of example, the first stage 210 may include a process of receiving source materials from a supplier for producing one or more of first component 12, second component 14, third component 16 of product 10. The second stage 220 may include a process of manufacturing first component 12, second component 14 (e.g., second component 14A, second component 14B), third component 16, and/or sensing devices 18, and/or assembling product 10. In embodiments in which product 10 includes a syringe storing a fluid substance, the second stage 220 may involve a process of filling the fluid substance within the syringe. The third stage 230 may include a process of testing the assembled product 10, such as from a quality control standpoint, to evaluate a proper assembly and operation of product 10. It should be appreciated that the third stage 230 may involve testing product 10 to an extent suitable for, and/or in accordance with an applicable standard and/or protocol associated with, the type of product.

The fourth stage 240 may include a process of sterilizing product 10 via a sterilization method. In some embodiments, the fourth stage 240 may involve a terminal sterilization of product 10 by a sterilization method using chemicals (e.g., vaporized hydrogen peroxide (VHP), ethylene oxide, nitrogen dioxide, etc.) to remove contaminants and other biological agents present on product 10. For example, the fourth stage 240 may include positioning product 10 within a sterilization chamber configured to run sterilization cycles at predefined temperatures and pressures, and supplying sterilizing chemicals into the sterilization chamber during the cycles at adjustable concentrations. It should be understood that the term “sterilization” refers to achieving a level of sterility appropriate for product 10. In the example of product 10 including an auto-injector storing a formulated drug substance, the fourth stage 240 may include the applicable sterilization techniques for commercial distribution and use of a medical device. It should be appreciated that the fourth stage 240 may involve cleaning product 10 to an extent suitable for, and/or in accordance with an applicable standard and/or protocol associated with, the type of product.

Still referring to FIG. 4, the fifth stage 250 may include a process of packaging product 10. For example, product 10 may be packaged within a protective container that is configured to house product 10, such as a blister packaging. Additionally and/or alternatively, the fifth stage 250 may include a process of labeling/marking product 10 or a packaging of product 10. The sixth stage 260 may include a process of storing product 10, such as upon completion of the aforementioned manufacturing, testing, sterilization, and packaging processes of product 10. Product 10 may be stored in isolation or with a plurality of other products, such as in containers suitable for transporting products to a retailer for sale. At the seventh stage 270, product 10 may be shipped by a distributor to one or more locations for sale and/or use. Product 10 may be shipped via various suitable transportation means (e.g., air, ground, sea, etc.) and upon reaching its destination, product 10 may be sold to an end user (e.g., a consumer) at the eight stage 280 and subsequently delivered to the end user at the ninth stage 290.

It should be understood that product 10 may interact with various and/or numerous objects, surfaces, or personnel during each of the plurality of stages of process 200. It should be understood that the term “interact” may include any physical, chemical, or other contact or exposure (direct or indirect) with product 10. For example, one or more of the plurality of components of product 10 (see FIG. 1) may be physically handled, controlled, positioned, and/or manipulated to different configurations and/or arrangements relative to one another, or a surrounding environment, during one or more of the plurality of stages of process 200. For further example, the components of product 10 may be exposed to various elements without experiencing physical contact, such as a light (or lack thereof), a heat (or lack thereof), a fluid (e.g., air, water, gas), and more during the plurality of stages of process 200. Additionally and/or alternatively, a packaging that product 10 may be disposed within may interact with various objects, surfaces, or personnel during the stages of process 200. In this instance, product 10 may be indirectly interacted with through the packaging of product 10.

One or more of the plurality of stages of process 200 may involve product 10 (and particularly one or more of first component 12, second component 14A, 14B, or third component 16) interacting with an object responsible for performing operations associated with the manufacture, quality control testing, sterilization, packaging, storage, shipment, sale, and/or delivery of product 10. The object may include, but is not limited to, one or more surfaces, tools, assemblies, machinery, equipment, instrumentation, systems, etc. In some embodiments, the object may be remotely controlled through an automated operation, such as by a computer. In other embodiments, the object may be manually controlled and operated by personnel in process 200.

For example, product 10 may interact with one or more personnel (e.g. humans) during one or more of the plurality of stages of process 200, such as the personnel responsible for performing operations associated with the manufacture, quality control testing, sterilization, packaging, storage, shipment, sale, or delivery of product 10. It should be appreciated that various other elements, factors, or influences may be present in process 200 and involved in one or more of the manufacture, quality control testing, sterilization, packaging, storage, shipment, sale, and delivery of product 10. That is, product 10 may have additional and/or alternative interactions with its surrounding environment during process 200 than those described herein without departing from a scope of this disclosure.

In either instance, an interaction between product 10 and an object, personnel, or other factor may cause the occurrence of an event during which product 10 may experience or encounter a parameter (e.g., a force, a pressure, a velocity, a tilt, a rotation, etc.) at a particular stage of process 200. Stated differently, the interaction of product 10 with its surrounding environment during one or more of the plurality of stages of process 200 may involve an event in which a change in (and/or creation of) a parameter occurs to at least one of the components of product 10, the parameter being sufficient to cause possible damage (e.g., structural, mechanical, chemical, etc.) to product 10. As described herein, sensing devices 18 may be configured to detect and/or measure the change and/or creation of the parameter to product 10 upon the occurrence of the event at the particular stage of process 200.

In some embodiments, a plurality of products 10 may undergo process 200 such that a plurality of parameters may be detected/measured upon the occurrence of a plurality of events at various stages of process 200. In this instance, an average of the parameters (e.g., a force) encountered by the plurality of products 10 may be determined and/or a range of the parameters encountered by products 10 during process 200 may be determined.

FIG. 5 depicts a flow diagram of an exemplary method 300 for identifying an occurrence of an event to a product during a supply chain process according to the present disclosure. It should be appreciated that the steps of method 300 are described herein may be executed by one or more users. In other embodiments, the steps of method 300 may be completed in the context of computer-executable instructions that may be executed or implemented by a remote computing system, such as one or more remote systems or computers that are in communication with sensing devices 18.

At step 302, a preparation and/or management of a product, such as product 10, may be performed at a current stage of a supply chain process, such as process 200. Stated differently, product 10 may be prepared and/or managed pursuant to the operations associated with any one of the plurality of stages of process 200 shown and described above (see FIG. 4) at step 302.

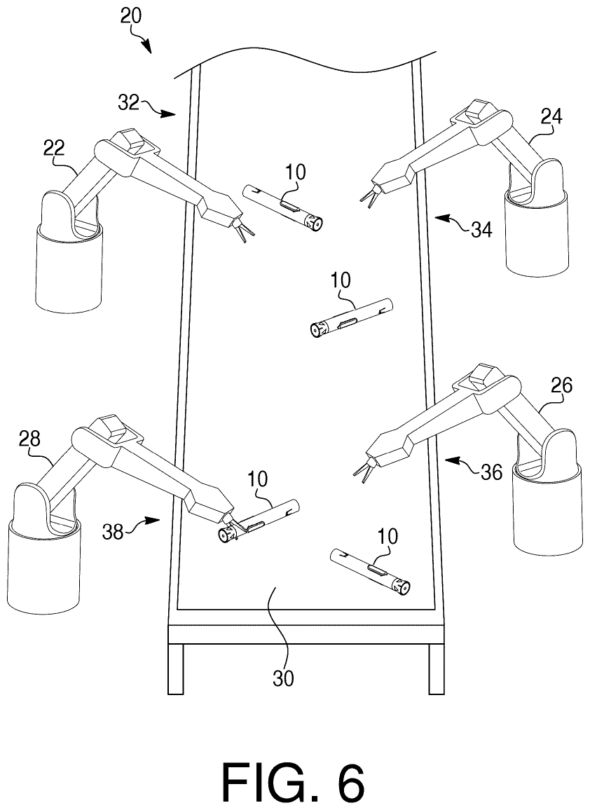

By way of example only, the current stage of process 200 at step 302 may include a manufacturing process of product 10 at the second stage 220 described above. For illustrative purposes, as seen in FIG. 6, a manufacturing environment 20 of the second stage 220 is depicted in which environment 20 may include a plurality of equipment positioned along various locations on an assembly line 30. For example, environment 20 may include a first manufacturing instrument 22 positioned adjacent to a first location 32 of the assembly line 30, a second manufacturing instrument 24 positioned adjacent to a second location 34 of the assembly line 30, a third manufacturing instrument 26 positioned adjacent to a third location 36 of the assembly line 30, and a fourth manufacturing instrument 28 positioned adjacent to a fourth location 38 of the assembly line 30. Although a particular number and arrangement of equipment is shown and described herein for the second stage 220, it should be understood that the manufacturing environment 20 is merely illustrative such that additional and/or fewer instruments (or other types of equipment) may be included in the second stage 220 of process 200, and in other suitable configurations, without departing from a scope of this disclosure.

In the example, each of the instruments in manufacturing environment 20 may be configured and operable to perform one or more operations at the respective location of the assembly line 30 for manufacturing product 10. During the second stage 220, product 10 (and particularly one or more of first component 12, second component 14A, 14B, and third component 16) may interact with various objects (e.g., first manufacturing instrument 22, second manufacturing instrument 24, third manufacturing instrument 26, and fourth manufacturing instrument 28) and/or surfaces (e.g., a portion of assembly line 30 along first location 32, second location 34, third location 36, and fourth location 38). Although not shown, it should be appreciated that manufacturing environment 20 may include various other elements or factors that may interact with product 10 during the manufacturing process of the second stage 220 (e.g., a light, a temperature, a fluid, etc.).

Referring back to FIG. 5, at step 304, whether the occurrence of an event is detected at the current stage of process 200, such as by sensing devices 18, is determined. As described in detail above, an event may be defined by an interaction (e.g., physical or chemical) between product 10 and the surrounding environment at the current stage of process 200, such as with an object, a personnel, or other factor. Occurrence of the event may cause one or more of the pair of sensing devices 18 to detect a change in (and/or creation of) a parameter applied to product 10. That is, the occurrence of the event may cause sensing devices 18 to detect a change in a property of product 10. The parameter may be to an extent or degree sufficient to cause a change in product 10 and/or at least partially impair product 10, such as by causing a defect or (structural) damage to one or more components of product 10.

For example, sensing devices 18 may be configured to detect an occurrence of an event to product 10 at the current stage of process 200 in response to one of the pair of sensing devices 18 moving and/or becoming dislodged within second component 14A, 14B (see FIGS. 2-3), as described in detail above. The occurrence of the event, and particularly a movement of sensing devices 18 within second component 14A, 14B, may be determined at step 304 in response to the visual inspection of second component 14A, 14B by the user(s).

By positioning the pair of sensing devices 18 within product 10, and particularly inside second component 14A, 14B, sensing devices 18 may be configured and operable to accurately detect the occurrence of an event experienced by product 10, and particularly a parameter applied thereto. For example, one or more sensing devices 18 positioned on an exterior surface of second component 14A, 14B may not accurately detect a parameter applied thereto, due to second component 14A, 14B being disposed inside an outer device body, such as third component 16 (e.g. a plastic auto-injector), and/or placed within a packaging that stores product 10. In some instances, with the outer device body (e.g., third component 16) and/or the packaging being formed of a material (e.g. plastic) that is different than second component 14A, 14B (e.g., glass), an accuracy of sensing devices 18 positioned on the exterior surface of second component 14A, 14B for detecting the parameter applied to product 10 may be decreased.

Additionally, sensing devices 18 placed on an exterior of the second component 14A, 14B may generate and/or form an exterior interface along second component 14A, 14B that does not interact and/or behave similarly with a surrounding environment of second component 14A, 14B during the stages of process 200. This may be due to the material of sensing devices 18 being different than the material of second component 14A, 14B (e.g. glass), which may impact an ability of sensing devices 18 to accurately detect a parameter applied thereto. For example, second component 14A, 14B may interact with a surrounding environment in a substantially different manner when sensing devices 18 are coupled to the exterior of second component 14A, 14B than when second component 14A, 14B does not include any sensing devices 18 secured to the exterior surface, due to sensing devices 18 being formed of a different material than second component 14A, 14B (e.g., glass).

By way of further example, sensing devices 18 may generate and/or form a cross-sectional profile of second component 14A, 14B that is different (e.g., relatively greater) than an original cross-sectional profile of second component 14A, 14B. As such, a size, a shape, and/or a configuration of a packaging of product 10 and/or of an outer device body (e.g., third component 16) that receives second component 14A, 14B may require modification to account for an increased cross-sectional profile of second component 14A, 14B, which may impair sensing devices 18 from accurately detecting a parameter applied thereto.

In response to determining an event did not occur during the current stage of process 200 at step 304, such as upon a visual inspection of product 10 by the user(s), method 300 may continue to step 306 in which product 10 continues to a next stage of process 200. In this instance, given that the current stage of process 200 was performed and completed without the occurrence of an event, method 300 may return to step 302 during which continued preparation and/or management of product 10 may be performed in accordance with the corresponding operations of the next stage of process 200. Alternatively, in response to determining that an event did occur during the current stage of process 200 at step 304, as detected by a visual inspection of the relative positions of sensing devices 18 within second component 14A, 14B, information and/or data associated with the event may be determined at steps 208 and 210.

The information and/or data may include a plurality of information, such as, but not limited to, sensor data indicative of the parameter(s) detected by one or more of the pair of sensing devices 18. In some embodiments, the sensor data may include a corresponding time period during the current stage of process 200 at which the parameter(s) was detected, such as from the timing recorded by the timer device of sensing devices 18, as described in detail above.

For illustrative example only, referring back to FIG. 6, the event may include a physical manipulation of product 10 (e.g., grasping) by fourth manufacturing instrument 28 while product 10 is positioned adjacent to the fourth location 38 of assembly line 30 in manufacturing environment 20. In grasping product 10, one or more of the pair of sensing devices 18 may detect a load (i.e. the parameter) applied to second component 14 (see FIG. 1) by fourth manufacturing instrument 28.

Referring to FIG. 5, at step 308, an originating source or cause of the event may be determined, such as based on the sensor data detected by sensing devices 18. In the example, the originating source or cause of the event (e.g. physical manipulation of product 10) may be determined to be the fourth manufacturing instrument 28 positioned adjacent to the fourth location 38 along the assembly line 30 in response to the timing recorded by the timer device. For example, the timer device may have ceased recording time upon the occurrence of the event, with the recorded time lapse indicating product 10 was located at the fourth location 38 along manufacturing environment 20 when the timing stopped. In other embodiments, product 10 may be visually inspected as fourth manufacturing instrument 28 encounters product 10 at the fourth location 38, thereby revealing it as the source of the event occurrence.

At step 310, a relative value or degree of the parameter applied to product 10 (e.g., the load) may be determined. For example, and as described above, sensing devices 18 may be configured to move and/or become dislodged upon application of a parameter having at least a minimum value. In other words, the parameter may be relative to a threshold that defines a maximum (acceptable) value or degree of the parameter without causing sensing devices 18 to move. In this instance, application of a parameter that causes sensing devices 18 to move is determined to exceed the threshold, such that the parameter may be determined to be at least greater than the maximum value or degree defining the threshold. As described above, a value or degree of a parameter that exceeds the threshold may be indicative of a rejection parameter that is sufficient to cause a change to the product and/or at least partially impair product 10 in some form, such as a structural change to the product, a functional change to the product, a weakening of the product, a change in a level of sterility of the product, a change in a level of fluid stored in the product, and more.

For example, the threshold may define a maximum value or degree of a parameter that corresponds to conditions in which damage or defect(s) (e.g., physical, functional, aesthetic, operational, etc.) to product 10 do not occur despite the occurrence of the event at step 304. In this instance, a parameter that is determined to exceed the threshold, in response to a visual confirmation of sensing devices 18 being moved and/or dislodged within product 10, may be indicative of a product that has experienced (or has an increased likelihood of experiencing) some form of damage or defect, while a parameter that does not exceed the threshold may be indicative of a product that has not experienced (or does not have an increased likelihood of experiencing) damage or defect.

It should be appreciated that the parameter determined at step 310 may be sufficient to cause a change or render product 10 at least partially damaged, defective, impaired, unsterile, and/or inoperable for its intended use, with the value or degree of the determined parameter being greater than the threshold. In examples in which product 10 may include a medical device, such as a syringe or an auto-injector having one or more of a needle, a plunger, and/or a barrel storing a fluid or a solid or a gel substance therein, a parameter applied to the medical device that exceeds the threshold may cause an inadvertent movement of said components. For example, the application of a parameter exceeding the threshold may involve deflecting the needle and rendering the medical device inoperable for use by a user (e.g., a patient), moving the plunger and causing a premature release of the stored fluid or gel or solid substance, or breaking the barrel and causing the stored fluid or solid or gel substance to leak therefrom. By way of further example, the application of a parameter exceeding the threshold may involve exposing the barrel to excessive vibration, shock, light and/or heat, thereby physically and/or chemically altering the properties of the substance stored therein.

In some embodiments, at least one threshold may be defined and/or predetermined for a plurality of parameters (e.g., a force, a pressure, a compression, a velocity, a tilt, a rotation, a shock, etc.) that may be experienced by product 10 in process 200. Accordingly, the applicable threshold corresponding to a type of parameter (from the plurality of parameters) determined at step 310 may be identified. It should be appreciated that the threshold may include a value or degree that corresponds to a unit of measurement of the associated parameter.

Still referring to FIG. 5, in response to determining the parameter exceeds the threshold at step 310, one or more proposed modifications to the current stage of process 200 may be identified at step 312, such as by the one or more user(s). The one or more proposed modifications identified at step 312 may be directed to resolving human or automated equipment error involved in process 200 at the current stage. For example, the one or more user(s) may be able to identify potential modifications to a position, a location, an operation, and/or various other characteristics of the originating source of the event for purposes of inhibiting further occurrences of the event (detected at step 304) with further products 10 during the current stage of process 200 due to the inclusion of sensing devices 18 in product 10. Referring to the illustrative example of FIG. 6, for example, the user(s) may be able to identify modifications to one or more characteristics of the fourth manufacturing instrument 28, which was determined to be the source of the event at step 308.

In another example, the one or more user(s) may be able to identify potential modifications to objects other than the source of the event which may directly and/or indirectly influence occurrence of the event. Referring to the illustrative example of FIG. 6, for example, the user(s) may be able to identify modifications to one or more of first manufacturing instrument 22, second manufacturing instrument 24, third manufacturing instrument 26, assembly line 30, and/or one or more other surfaces, tools, assemblies, machinery, equipment, instrumentation, systems, etc.

As described in detail above, one or more objects responsible for performing operations associated with a current stage of process 200 (e.g., manufacturing, quality control testing, sterilization, packaging, storage, shipment, sale, and/or delivery) may be controlled through an automated operation (e.g., by a computer). In this instance, the potential modification(s) identified at step 312 may include adjustments to the automated control of the object, such as by modifying computer-executable instructions and/or programmable computer code operable to automate operation of the object. In embodiments in which the object may be manually operated by personnel of process 200, the potential modification(s) identified at step 312 may be communicated to the user and/or personnel for implementation.

By identifying the occurrence of the event during process 200, a user may be allowed to implement the one or more modifications to process 200. It should be appreciated that method 300 may allow the user(s) to ensure an effectiveness of the various stages of process 200 and/or of a packaging of product 10 when manufacturing product 10. Method 300 may identify any stages along process 200 that are sources for potential inefficiencies in manufacturing product 10, such as due to a mishandling of product 10.

FIG. 7 depicts another exemplary second component 50 of product 10 of FIG. 1. It should be appreciated that second component 50 may be incorporated into product 10 in lieu of second component 14A, 14B shown and described above without departing from a scope of this disclosure. In the example, second component 50 may include a vial having a body 52 with a longitudinal length defined between a proximal end 54 and a distal end 56. Second component 50 may include an internal cavity defined by body 52 between proximal end 54 and distal end 56, and an opening 58 at distal end 56 that facilitates access into the internal cavity of body 52. Body 52 may be formed of a (second) material that is different than the first material of the remaining components of product 10, such as glass and/or plastic.

Second component 50 may include a base 60 that is sized, shaped, and/or otherwise configured to attach to body 52 at distal end 56. It should be appreciated that base 60 may enclose the internal cavity of body 52 when removably coupled to distal end 56. In other words, base 60 may define an enclosed bottom end of body 52 when attached to distal end 56. Body 52 and base 60 of second component 50 may be formed of a same material, such that base 60 may maintain the same coefficient of friction as body 52. Base 60 may include one or more attachment mechanisms in the form of a pair of tracks 62 extending proximally (e.g., upwards) from base 60. Each of the pair of tracks 62 may be positioned along base 60 in an opposed configuration relative to one another, such that each track 62 may be aligned with and facing one another. In the example, base 60 may include two pairs of tracks 62 that are offset from one another at a 90° (degree) angle from each other. Accordingly, base 60 may include four tracks 62. It should be appreciated that base 60 may include additional and/or fewer tracks 62 than those shown and described herein without departing from a scope of this disclosure.

Still referring to FIG. 7, each track 62 may include one or more openings and/or sockets 64 positioned along a longitudinal length of the respective track 62. In the example, each track 62 may include four sockets 64 extending between opposing ends of the respective track 62 at predefined intervals and/or positions. Accordingly, tracks 62 may define a plurality of locations at varying heights for assembling sensing devices 18 within second component 50 based on the relative anchor points defined by sockets 64 on tracks 62. It should be appreciated that tracks 62 may include additional and/or fewer sockets 64 than those shown and described herein without departing from a scope of this disclosure. Sockets 64 may define a plurality of attachment positions for sensing devices 18 relative to each track 62.

The one or more sockets 64 may be sized, shaped, and/or otherwise configured to receive at least a portion of retention mechanism 19 for securely coupling sensing device 18 between a pair of opposing tracks 62. Sensing device 18 may be biased radially-outwards by biasing mechanism 17, as described in detail above, such that sensing device 18 may be fixedly coupled between opposing tracks 62 in response to retention mechanisms 19 being urged outwards towards the opposing tracks 62 and into a respective socket 64, thereby maintaining sensing device 18 at a fixed position relative to tracks 62.

In this instance, tracks 62 may provide an attachment mechanism for securely coupling one or more sensing devices 18 to base 60 such that, upon attaching base 60 to body 52 and extending tracks 62 into the internal cavity via opening 58, sensing devices 18 may be maintained in the fixed position between the opposing tracks 62 and disposed within the internal cavity of second component 50. In other words, body 52 of second component 50 may be coupled to base 60 after one or more sensing devices 18 are preloaded onto and secured in tracks 62. Body 52 may be selectively coupled to base 60 via various suitable mechanisms, including but not limited to, an adhesive, a snap-fit connection, and more. It should be appreciated that base 60, and particularly tracks 62, may provide an enhanced attachment interface for securing sensing devices 18 within second component 18 to maintain a secured engagement between retention mechanisms 19 and the internal surface of body 52.

It should be understood that tracks 62 may be configured to enhance a connection between sensing devices 18 and the interior surface of body 52. For example, in embodiments in which body 52 may be formed of glass or other material, it should be appreciated that retention mechanisms 19 may experience slippage and/or inadvertent movement against the interior surface of body 52 due to the properties of the interior surface. In this instance, sensing devices 18 may move upon the application of a parameter (e.g., a force) of a value or degree that does not exceed the minimum threshold. In this instance, sensing device 18 may detect the occurrence of an event that is not indicative of a product that has experienced (or has an increased likelihood of experiencing) some form of damage or defect. Accordingly, the attachment mechanism defined by tracks 62 and sockets 64 may be collectively configured to include one or more interfaces along the interior surface of body 52 for securely coupling sensing devices 18 within second component 50. In this instance, the attachment mechanism may allow for accurately detecting the occurrence of an event upon sensing devices 18 becoming dislodged from tracks 62.

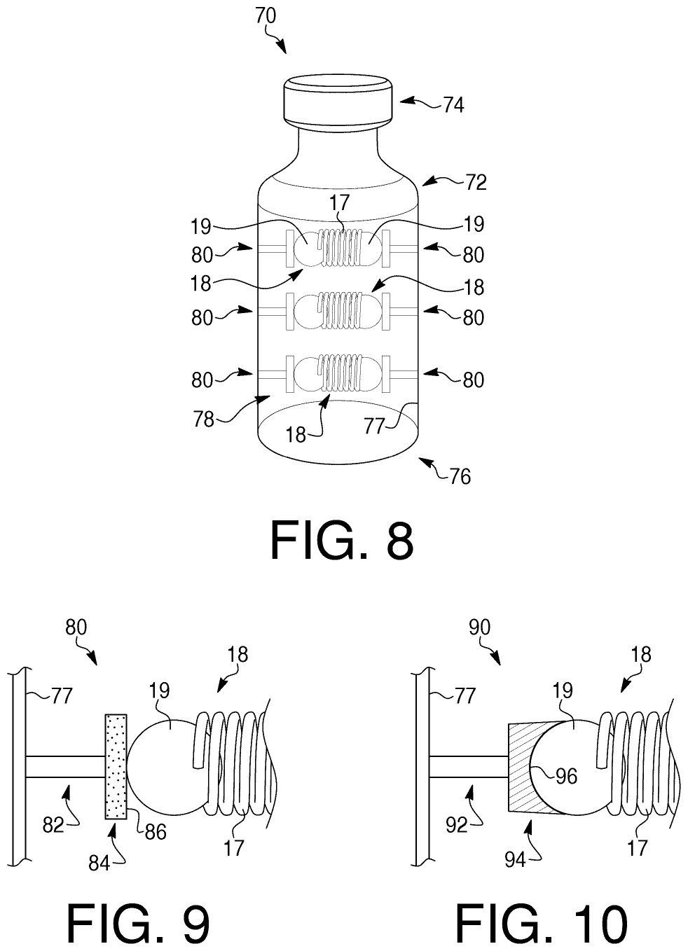

FIG. 8 depicts another exemplary second component 70 of product 10 of FIG. 1. It should be appreciated that second component 70 may be incorporated into product 10 in lieu of second component 14A, 14B shown and described above without departing from a scope of this disclosure. In this example, second component 70 may include a vial having a body 72 with a longitudinal length defined between a proximal end 74 and a distal end 76. Second component 70 may include an internal cavity 78 defined by body 72 between proximal end 74 and distal end 76 for storing the fluid substance. Body 72 may be formed of a (second) material, such as glass and/or plastic. Second component 70 may include a plurality of attachment mechanisms 80 disposed within internal cavity 78, and particularly against an interior surface 77 of body 72. As described herein, the plurality of attachment mechanisms 80 may be collectively configured to securely couple a plurality of sensing devices 18 to body 72.

In the example, second component 70 may include three pairs of opposing attachment mechanisms 80 secured to an interior surface 77 of body 72 and three corresponding sensing devices 18 securely coupled within internal cavity 78 between each pair of opposing attachment mechanisms 80. It should be appreciated that additional and/or fewer attachment mechanisms 80 may be included in body 72 for securely coupling a corresponding number of sensing devices 18 within second component 70 without departing from a scope of this disclosure. It should further be appreciated that attachment mechanisms 80 may be positioned within body 72 at various other suitable locations, arrangements, and/or orientations between proximal end 74 and distal end 76 and/or relative to one another within internal cavity 78.

Still referring to FIG. 8, attachment mechanisms 80 may be positioned at preset locations along a longitudinal length of body 72 on interior surface 77, such as between proximal end 74 and distal end 76. Sensing devices 18 may be coupled to interior surface 77 via one or more pairs of attachment mechanisms 80. Each pair of attachment mechanisms 80 may be positioned within body 72 in an opposed configuration relative to one another. The pair of attachment mechanisms 80 may be placed in parallel alignment relative to one another such that at least one sensing device 18 may be disposed between the pair of opposing attachment mechanisms 80. Sensing devices 18 may be biased radially-outwards as described in detail above, such that sensing devices 18 may be fixedly coupled between the pair of attachment mechanisms 80 in response to retention mechanism 19 engaging attachment mechanisms 80.

FIG. 9 depicts at least one attachment mechanism 80 coupled and/or attached to interior surface 77. In the example, attachment mechanism 80 may include a stem 82 coupled to and extending laterally-inwards from interior surface 77, and a receiver 84 coupled to a terminal end of stem 82. Stem may have a longitudinal length that is arranged in a transverse alignment relative to a longitudinal length of body 72, and receiver 84 may have a longitudinal length with a planar surface 86 that is arranged in a transverse alignment relative to a longitudinal length of stem 82. In other words, stem 82 may extend transversely (e.g., perpendicularly) relative to interior surface 77 and receiver 84 may extend parallel to interior surface 77. In this instance, attachment mechanism 80 may generally form a T-shaped configuration between stem 82 and receiver 84.

Planar surface 86 may define an interface for engaging retention mechanism 19, such that receiver 84 may be configured to couple sensing device 18 to attachment mechanism 80. In other words, retention mechanism 19 may be configured to abut against planar surface 86 of receiver 84, thereby securely attaching retention mechanism 19 to interior surface 77 via attachment mechanism 80. In some embodiments, planar surface 86 may include one or more exterior surface features that is configured to provide a frictional interference with retention mechanism 19. In other embodiments, planar surface 86 may be formed of a material configured to increase the frictional resistance against retention mechanism 19. In either instance, planar surface 86 may be configured to at least partially inhibit movement and/or slippage of sensing device 18 relative to receiver 84 when in contact with retention mechanism 19.

Although not shown in FIG. 9, it should be appreciated that the opposing retention mechanism 19 of the particular sensing device 18 may be coupled to an opposing attachment mechanism 80 positioned along the opposing interior surface 77 of body 72, in a substantially similar manner as shown and described above. Accordingly, each sensing device 18 may be coupled within second component 70 between a pair of opposing attachment mechanisms 80 at a location within body 72 corresponding to the relative positions of the pair of attachment mechanisms 80. As shown in FIG. 8, a plurality of attachment mechanisms 80 may be coupled at various heights and/or locations along interior surface 77 for securing sensing devices 18 within body 72.

FIGS. 10-12 depict additional exemplary attachment mechanisms of second component 70 for enhancing a grasping force of one or more sensing devices 18 disposed therein. It should be appreciated that the attachment mechanisms described herein may be incorporated into second component 70 in lieu of attachment mechanism 80 shown and described above without departing from a scope of this disclosure. Although a single attachment mechanism is described and depicted in FIGS. 10-12, it should be appreciated that such depiction is for illustrative purposes only and that second component 70 may include a plurality of attachment mechanisms, and at least one opposing attachment mechanism within body 72 from those depicted in FIGS. 10-12 for securely coupling at least one sensing device 18 between the pair of attachment mechanisms.

Referring initially to FIG. 10, for example, an exemplary attachment mechanism 90 is depicted. Attachment mechanism 90 may be attached to interior surface 77 of second component 70. Attachment mechanism 90 may include a stem 92 that is sized, shaped, and/or otherwise configured in a substantially similar manner as stem 82 shown and described above. Attachment mechanism 90 may further include a receiver 94 positioned on a terminal end of stem 92. Receiver 94 may include a concave (cup-like) surface 96 that is sized, shaped, and/or otherwise configured to receive a portion of retention mechanism 19 of sensing device 18 therein. In other words, concave surface 96 may define a cavity and/or enclosed opening on receiver 94 that corresponds to a cross-sectional profile of retention mechanism 19. In this instance, concave surface 96 may define an interface on receiver 94 for securely engaging retention mechanism 19, thereby coupling sensing device 18 to interior surface 77 via attachment mechanism 90.

Although receiver 94 is shown and described herein as including concave surface 96, it should be appreciated that receiver 94 may include various suitable sizes, shapes, and/or configurations without departing from a scope of this disclosure. Although not shown, in other embodiments retention mechanisms 19 may include one or more features and/or surfaces that may be sized, shaped, and/or otherwise configured to interface with receiver 94. For example, an exterior surface of retention mechanism 19 may include a socket that is configured to mate with receiver 94 to securely couple sensing device 18 to attachment mechanism 90. In some embodiments, the socket may be generally V-shaped.

Referring now FIG. 11, another exemplary attachment mechanism 100 is depicted in which attachment mechanism 100 may be attached to interior surface 77 of second component 70. Attachment mechanism 100 may include a stem 102 that is sized, shaped, and/or otherwise configured in a substantially similar manner as stem 82 shown and described above. Attachment mechanism 100 may further include a receiver 104 positioned on a terminal end of stem 102. Receiver 104 may include a recess 106 disposed between two opposing arms (e.g., prongs) 108 extending laterally outward on opposing sides of recess 106. Recess 106 may be sized, shaped, and/or otherwise configured to receive a portion of retention mechanism 19 therein, and opposing arms 108 may be sized, shaped, and/or otherwise configured to abut against one or more portions of retention mechanism 19. In some embodiments, opposing arms 108 may be configured to guide retention mechanism 19 towards recess 106, thereby maintaining retention mechanism 19 in engagement with receiver 104. In other words, recess 106 and opposing arms 108 may collectively define an interface on receiver 104 for securely engaging retention mechanism 19, thereby coupling sensing device 18 to interior surface 77 via attachment mechanism 100.

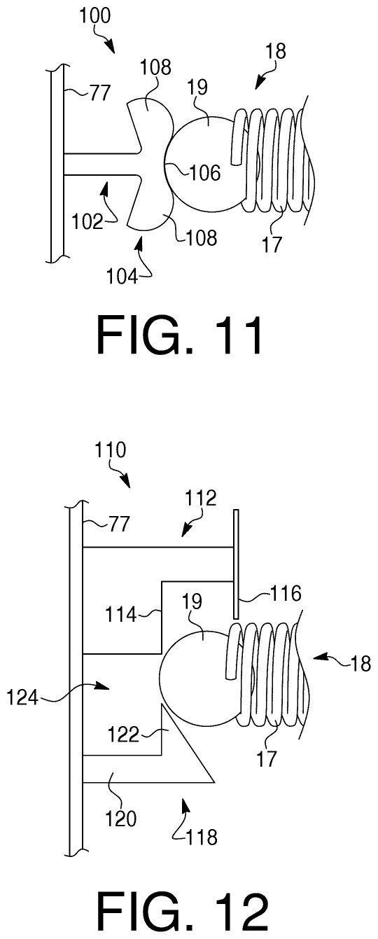

FIG. 12 depicts another exemplary attachment mechanism 110 extending outwards from interior surface 77 of second component 70. Attachment mechanism 110 may include a first arm 112 and a second arm 118 that are each secured to interior surface 77. In the example, first arm 112 may be generally L-shaped and include a base surface 114 and an overhang surface 116 disposed at least partially over base surface 114. Second arm 118 may include a stem 120 that extends laterally outwards from interior surface 77 and a hook 122 positioned on a terminal end of stem 120 that extends at least partially inwards towards interior surface 77. In the example, hook 122 may be sized and/or shaped with an angled configuration that extends towards first arm 112, and particularly base surface 114. The pair of arms 112, 118 may collectively define an intermediate space 124 disposed there between for receiving retention mechanism 19 therein.

In the example, one or more of hook 122 and/or base surface 114 may be configured to contact and/or abut against retention mechanism 19 when coupling sensing device 18 to attachment mechanism 110. In this instance, the angled configuration of hook 122 may guide retention mechanism 19 towards first arm 112, upon retention mechanism 19 encountering an exterior surface of hook 122, until encountering base surface 114. Overhang surface 116 may be configured to at least partially engage retention mechanism 19 and inhibit lateral movement of retention mechanism 19 away from base surface 114. One or more of arms 112, 118 may be at least partially flexible and/or movable relative to one another or interior surface 77. In the example, second arm 118 may be configured to move (e.g., deflect) in response to retention mechanism 19 of sensing device 18 being received between first arm 112 and second arm 118 to allow receipt of retention mechanism 19 into intermediate space 124 disposed between arms 112, 118.

In this instance, second arm 118 may deflect away from first arm 112, such as in response to biasing mechanism 17 urging retention mechanism 19 between arms 112, 118. In this instance, as second arm 118 is deflected away from first arm 112, retention mechanism 19 may extend into intermediate space 124 between arms 112, 118. With retention mechanism 19 disposed within intermediate space 124, second arm 118 may move towards first arm 112 and return to an original position. In this instance, hook 122 may be configured to engage retention mechanism 19, thereby securing retention mechanism 19 in a fixed position against interior surface 77 and between arms 112, 118.

It should be appreciated that the general discussion of this disclosure provides a brief, general description of a suitable computing environment in which the present disclosure may be implemented. In one embodiment, any of the disclosed systems and/or methods may be executed by or implemented by a computing system consistent with or similar to that explained in this disclosure (e.g., one or more remote systems). Although not required, aspects of the present disclosure are described in the context of computer-executable instructions, such as routines executed by a data processing device, e.g., a computer hardware platform, a wireless device, and/or a personal computer. Those skilled in the art will appreciate that aspects of the present disclosure can be practiced with other communications, data processing, or computer system configurations. Indeed, the terms “computer,” “processor,” “remote systems” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain steps of method 300, are described as being performed exclusively on a single device or system, the present disclosure may also be practiced in distributed environments where functions or modules are shared among disparate processing devices, which are linked through a communications network. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media. Alternatively, computer implemented instructions, data structures, screen displays, and other data under aspects of the present disclosure may be distributed over the Internet and/or over other networks (including wireless networks). Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, which may provide non-transitory storage at any time for the software programming. As used herein, unless restricted to non-transitory, tangible “storage” media, terms such as computer “readable medium” refer to any medium that participates in providing instructions to a processor for execution.

While a number of embodiments are presented herein, multiple variations on such embodiments, and combinations of elements from one or more embodiments, are possible and are contemplated to be within the scope of the present disclosure. Moreover, those skilled in the art will appreciate that the conception upon which this disclosure is based may readily be used as a basis for designing other devices, methods, and systems for carrying out the several purposes of the present disclosure.

Embodiments of the present disclosure may include the following features:

Item 1. A method for identifying an event during a supply chain process, comprising: preparing a product during the supply chain process; determining an occurrence of the event during preparation of the product during the supply chain process via a sensing device engaged against an interior surface of the product, wherein the event causes the sensing device to disengage the interior surface, thereby indicating a change to the product; and identifying a modification to the supply chain process to inhibit occurrences of the event during subsequent preparations of the product in the supply chain process.

Item 2. The method of item 1, further comprising: determining a source in the supply chain process causing the occurrence of the event, wherein identifying the modification includes identifying an adjustment to an operation of the source.

Item 3. The method of item 2, wherein prior to identifying the modification, the method comprises: determining a parameter is applied to the product during the event by the source, the parameter causing damage to the product.

Item 4. The method of item 3, wherein the sensing device is configured to detect the parameter applied to the product during the occurrence of the event.

Item 5. The method of item 4, wherein the sensing device is configured to move relative to the interior surface of the product in response to detecting the parameter during the occurrence of the event.

Item 6. The method of item 5, wherein the sensing device is coupled to the product within the internal cavity at a fixed position prior to application of the parameter, and dislodged from the fixed position upon application of the parameter to the product.

Item 7. The method of item 3, wherein the parameter includes a pressure, a force, a velocity, a compression, a tilt, or a rotation experienced by the product during the event.

Item 8. The method of item 4, wherein sensing device includes an accelerometer, a gyro sensor, or a pressure sensor.

Item 9. The method of item 2, wherein: the product includes a device, a tool, a machine, an instrument, a mechanism, an appliance, a gadget, or an apparatus; and the source includes a surface, a tool, an assembly, a machinery, an equipment, an instrumentation, or a system responsible for performing operations in the supply chain process to prepare the product.

Item 10. The method of item 9, wherein the product includes a medical device and the source includes an automated instrumentation for manufacturing the medical device, such that identifying the modification to the supply chain process includes an adjustment to computer-executable instructions defining an operation of the automated instrumentation.

Item 11. The method of item 10, wherein the medical device includes an autoinjector or a syringe.

Item 12. The method of item 11, wherein the autoinjector or the syringe includes a container or a vial, wherein the sensing device is disposed inside an interior cavity of the container or the vial.

Item 13. The method of item 9, wherein preparing the product during the supply chain process includes one or more of manufacturing, assembling, testing, sterilizing, packaging, labeling or marking, storing, shipping, selling, or delivering the product.

Item 14. The method of item 2, wherein the event is a first event occurring during a first stage of the supply chain process for preparing the product, and the source causing the occurrence of the event is a first source in the supply chain process for preparing the product at the first stage.

Item 15. The method of item 1, wherein the change includes a structural change to the product, a change in a level of sterility of the product, or a change in a level of substance stored in the product.

Item 16. The method of item 1, wherein the sensing device includes a biasing mechanism and a pair of retention mechanisms disposed at opposing ends of the biasing mechanism; wherein the biasing mechanism is biased towards an expanded configuration, such that the biasing mechanism is configured to urge the pair of retention mechanisms away from one another; and wherein each of the pair of retention mechanisms is configured to abut against the interior surface of the product, thereby coupling the sensing device to the product at a fixed position within the product.

Item 17. The method of item 16, wherein the sensing device is configured such that at least one of the pair of the retention mechanisms is configured to move relative to the interior surface upon the occurrence of the event to the product.

Item 18. The method of item 17, wherein the at least one of the pair of retention mechanisms is configured to move in response to the biasing mechanism compressing or expanding in response to the occurrence of the event, thereby dislodging the sensing device from engagement with the interior surface of the product.

Item 19. The method of item 1, wherein the product includes an attachment mechanism defining a base that is removably coupled to a body of the product; wherein the base includes a pair of tracks positioned on opposing sides of the base, the pair of tracks are received inside an internal cavity of the product and positioned along the interior surface when the base is coupled to the body of the product; wherein the pair of tracks are configured to couple with the sensing device such that the sensing device is disposed between the pair of tracks and positioned inside the internal cavity at a fixed position.

Item 20. The method of item 1, wherein the product includes a pair of attachment mechanisms disposed along opposing sides of the interior surface of the product for securely coupling the sensing device to the product.

Item 21. The method of item 20, wherein the pair of attachment mechanisms include a planar surface that is configured to engage and maintain the sensing device at a fixed position relative to the interior surface.

Item 22. The method of item 21, wherein the planar surface is formed of a material that is configured to generate a frictional resistance between the pair of attachment mechanisms and the sensing device.

Item 23. The method of item 20, wherein the pair of attachment mechanisms include a concave surface that is configured to engage and maintain the sensing device at a fixed position relative to the interior surface.

Item 24. The method of item 20, wherein the pair of attachment mechanisms include a recess disposed between a pair of arms, wherein the recess and the pair of arms are configured to engage and maintain the sensing device at a fixed position relative to the interior surface.

Item 25. The method of item 20, wherein the pair of attachment mechanisms each include a pair of arms configured to engage and maintain the sensing device at a fixed position relative to the interior surface, wherein at least one of the pair of arms is flexibly movable to allow receipt of the sensing device between the pair of arms.

Item 26. A method for reducing an occurrence of an event during a supply chain process, comprising: preparing a product during the supply chain process; determining the occurrence of the event while preparing the product in the supply chain process via a sensing device coupled against an interior surface of the product, wherein the event causes the sensing device to decouple the interior surface, thereby indicating a change to the product; and determining a modification to the supply chain process that reduces the occurrence of the event in the supply chain process during subsequent preparations of the product, thereby reducing the occurrence of the change to the product.

Item 27. The method of item 26, wherein prior to determining the modification, the method comprises: determining a source responsible for preparing the product in the supply chain process is causing the occurrence of the event; determining a parameter applied to the product by the source during the occurrence of the event exceeds a threshold; and determining the modification in response to the parameter exceeding the threshold.

Item 28. The method of item 27, wherein determining the source responsible for preparing the product in the supply chain process that is causing the occurrence of the event comprises: determining a timing in the supply chain process when the event occurs.

Item 29. The method of item 27, wherein the product includes a device, a tool, a machine, an instrument, a mechanism, an appliance, a gadget, or an apparatus; wherein the source includes a surface, a tool, an assembly, a machinery, an equipment, an instrumentation, or a system responsible for performing operations in the supply chain process to prepare the product; and wherein the parameter includes a pressure, a force, a velocity, a compression, a vibration, a shock, a tilt, or a rotation experienced by the product during the event.

Item 30. The method of item 26, wherein preparing the product during the supply chain process includes one or more of manufacturing, assembling, testing, sterilizing, packaging, labeling or marking, storing, shipping, selling, or delivering the product.

Item 31. The method of item 26, wherein the change to the product includes a structural change, a change in a level of sterility, or a change in a level of substance stored in the product.

Item 32. The method of item 26, wherein the sensing device is coupled against the interior surface of the product by engaging at least one attachment mechanism that is secured to the interior surface, and the sensing device is decoupled from the interior surface in response to disengaging the at least one attachment mechanism.

Item 33. A method for identifying an event causing a change to a product during a supply chain process, comprising: preparing the product during the supply chain process; determining the event occurs when preparing the product in the supply chain process in response to a sensing device disposed within an interior cavity of the product at a fixed position moving such that the product experiences the change during the supply chain process; and identifying a modification to the supply chain process that inhibits the change to the product by inhibiting an occurrence of the event during the supply chain process.

Item 34. The method of item 33, wherein prior to identifying the modification, the method comprises: determining a source of the event and a parameter applied to the product by the source that causes the sensing device to move within the interior cavity of the product, wherein the parameter exceeds a threshold sufficient for causing the change to the product.

Item 35. The method of item 34, wherein preparing the product during the supply chain process includes one or more of manufacturing, assembling, testing, sterilizing, packaging, labeling or marking, storing, shipping, selling, or delivering the product; and wherein the source includes a surface, a tool, an assembly, a machinery, an equipment, an instrumentation, or a system that performs operations in the supply chain process for preparing the product.

Item 36. The method of item 33, wherein the change to the product includes a structural change, a change in a level of sterility, or a change in a level of substance stored in the product.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for identifying an event during a supply chain process, comprising:
preparing a product during the supply chain process;
determining an occurrence of the event during preparation of the product during the supply chain process via a sensing device engaged against an interior surface of the product, wherein the event causes the sensing device to disengage the interior surface, thereby indicating a change to the product; and
identifying a modification to the supply chain process to inhibit occurrences of the event during subsequent preparations of the product in the supply chain process.

2. The method of claim 1, further comprising:
determining a source in the supply chain process causing the occurrence of the event, wherein identifying the modification includes identifying an adjustment to an operation of the source.

3. The method of claim 2, wherein prior to identifying the modification, the method comprises:
determining a parameter is applied to the product during the event by the source, the parameter changing or causing damage to the product.

4. The method of claim 3, wherein the sensing device is configured to detect the parameter applied to the product during the occurrence of the event.

5. The method of claim 4, wherein the sensing device is configured to move relative the interior surface of the product in response to detecting the parameter during the occurrence of the event.

6. The method of claim 5, wherein the sensing device is coupled to the product against the interior surface at a fixed position prior to application of the parameter, and dislodged from the fixed position upon application of the parameter to the product.

7. The method of claim 3, wherein the parameter includes a pressure, a force, a velocity, a compression, a tilt, or a rotation experienced by the product during the event.

8. The method of claim 4, wherein the sensing device includes an accelerometer, a gyro sensor, or a pressure sensor.

9. The method of claim 2, wherein:
the product includes a device, a tool, a machine, an instrument, a mechanism, an appliance, a gadget, or an apparatus; and
the source includes a surface, a tool, an assembly, a machinery, an equipment, an instrumentation, or a system responsible for performing operations in the supply chain process to prepare the product.

10. The method of claim 9, wherein the product includes a medical device and the source includes an automated instrumentation for manufacturing the medical device, such that identifying the modification to the supply chain process includes an adjustment to computer-executable instructions defining an operation of the automated instrumentation.

11. The method of claim 10, wherein the medical device includes an autoinjector or a syringe; and
wherein the autoinjector or the syringe includes a container or a vial, wherein the sensing device is disposed inside an interior cavity of the container or the vial.

12. The method of claim 9, wherein preparing the product during the supply chain process includes one or more of manufacturing, assembling, testing, sterilizing, packaging, labeling or marking, storing, shipping, selling, or delivering the product.

13. The method of claim 2, wherein the event is a first event occurring during a first stage of the supply chain process for preparing the product, and the source causing the occurrence of the event is a first source in the supply chain process for preparing the product at the first stage.

14. The method of claim 1, wherein the change includes a structural change to the product, a change in a level of sterility of the product, or a change in a level of substance stored in the product.

15. The method of claim 1, wherein the sensing device includes a biasing mechanism and a pair of retention mechanisms disposed at opposing ends of the biasing mechanism;
wherein the biasing mechanism is biased towards an expanded configuration, such that the biasing mechanism is configured to urge the pair of retention mechanisms away from one another; and
wherein each of the pair of retention mechanisms is configured to abut against the interior surface of the product, thereby coupling the sensing device to the product at a fixed position within the product.

16. The method of claim 15, wherein the sensing device is configured such that at least one of the pair of the retention mechanisms is configured to move relative to the interior surface upon the occurrence of the event to the product.

17. The method of claim 16, wherein the at least one of the pair of retention mechanisms is configured to move in response to the biasing mechanism compressing or expanding in response to the occurrence of the event, thereby dislodging the sensing device from engagement with the interior surface of the product.

18. The method of claim 1, wherein the product includes an attachment mechanism defining a base that is removably coupled to a body of the product;
wherein the base includes a pair of tracks positioned on opposing sides of the base, the pair of tracks are received inside an internal cavity of the product and positioned along the interior surface when the base is coupled to the body of the product; and
wherein the pair of tracks are configured to couple with the sensing device such that the sensing device is disposed between the pair of tracks and positioned inside the internal cavity at a fixed position.

19. The method of claim 1, wherein the product includes a pair of attachment mechanisms disposed along opposing sides of the interior surface of the product for securely coupling the sensing device to the product; and
wherein the pair of attachment mechanisms include:
a planar surface that is configured to engage and maintain the sensing device at a fixed position relative to the interior surface;
a concave surface that is configured to engage and maintain the sensing device at a fixed position relative to the interior surface;
a recess disposed between a pair of arms, wherein the recess and the pair of arms are configured to engage and maintain the sensing device at a fixed position relative to the interior surface; or
a pair of arms configured to engage and maintain the sensing device at a fixed position relative to the interior surface, wherein at least one of the pair of arms is flexibly movable to allow receipt of the sensing device between the pair of arms.

20. The method of claim 19, wherein the planar surface is formed of a material that is configured to generate a frictional resistance between the pair of attachment mechanisms and the sensing device.
